(19) United States
(12) Patent Application Publication
(10) Pub. No.: US 2010/0036202 A1
(43) Pub. Date: Feb. 11, 2010

(54) FOUR-DIRECTIONAL TIP DEFLECTION DEVICE FOR ENDOSCOPE

(75) Inventors: Wei-Teng Lin, Tainan City (TW); Chih-Chun Chan, Miaoli County (TW)

Correspondence Address: WPAT, PC;INTELLECTUAL PROPERTY ATTORNEYS, 2030 MAIN STREET, SUITE 1300, IRVINE, CA, 92614, US

(73) Assignee: Medical Intubation Technology Corporation, Taoyuan Hsien (TW)

(21) Appl. No.: 12/537,490

(22) Filed: Aug. 7, 2009

(30) Foreign Application Priority Data
Aug. 11, 2008 (TW) 097130581

(51) Int. Cl. A61B 1/01

(52) U.S. Cl. 600/146

(57) ABSTRACT

The present invention discloses a four-directional tip deflection device for endoscope including a tip deflecting mechanism, an elastic element, a support portion, a four-directionally flexible body, and first, second, third and fourth wires. The tip deflecting mechanism includes an endoscope lens, and first, second, third and fourth connecting portions facing the endoscope lens. The elastic element is sheathed to the tip deflecting mechanism. The support portion is sheathed to the elastic element. The four-directionally flexible body disposed in a first accommodating space has an end connected to the tip deflecting mechanism and another end connected to the support portion. The first, second, third and fourth wires are connected to the first, second, third and fourth connecting portions respectively. If the first, second, third or fourth wire is pulled, the four-directionally flexible body will be bent into an angle corresponding with a first, second, third or fourth wire direction respectively.

### BACKGROUND OF THE INVENTION

1. Field of the Invention

The present invention relates to a tip deflection device for endoscope, in particular to a four-directional tip deflection device for endoscope that is bendable to an angle corresponding with a first wire direction, a second wire direction, a third wire direction or a fourth wire direction by using a four-directionally flexible body installed in an elastic element.

2. Description of the Related Art

At present, four-directional tip deflection device for endoscope is used extensively for examining a digestive tract and organs. With reference to FIG. 1 for a schematic view of a conventional four-directional tip deflection device for endoscope, the four-directional tip deflection device for endoscope 91 includes a tip deflecting structure 92 installed at a front end of the four-directional tip deflection device for endoscope 91, a plurality of first metal rings 931, a plurality of second metal rings 932, and a plurality of rivets 94. Each first metal ring 931 includes a first rivet hole 951 and a second rivet hole 952 facing with one another, and each second metal ring 932 includes a third rivet hole 953 and a fourth rivet hole (not shown in the figure) facing with one another, such that each rivet 94 is nailed into each rivet hole to connect the first metal ring 931 and the second metal ring 932. If a first wire 961, a second wire 962, a third wire 963 or a fourth wire 964 wrapped in the plurality of metal rings is pulled to move the tip deflecting structure 92, the tip deflecting structure 92 will turn to a corresponding direction and a corresponding position to control the endoscope lens 921, such that the endoscope will enter into a human cavity such as a stomach for observing tissue images in a vast majority of the interior of the cavity.

However, the conventional four-directional tip deflection device for endoscope 91 requires a high dimensional precision below 0.2 mm for each metal ring, rivet hole and rivet, and thus incurs a complicated production procedure, a high manufacturing cost, and a high price to maintain a high-precision structure.

Due to the limitations of the structure of each metal ring of the conventional four-directional tip deflection device for endoscope, the bending angle will be limited and fixed, so that the conventional device cannot achieve a large bending angle or observe tissue images inside the cavity completely, and a missing part of a certain tissue image in the cavity may result, so that the application of the conventional four-directional tip deflection device for endoscope is substantially limited.

Furthermore, each bent first or second metal ring cannot resume its original position, but remains at the pulled angle, after the first, second, third or fourth wire is released and loosened. Users have to pull the corresponding first wire, second wire, third wire or fourth wire back to its original position, and thus the conventional four-directional tip deflection device for endoscope causes tremendous inconvenience to users.

In view of the aforementioned shortcomings of the prior art, the inventor of the present invention based on years of experience in the related industry to conduct extensive researches and experiments, and finally developed a four-directional tip deflection device for endoscope in accordance with the present invention to overcome the shortcomings of the prior art.

### SUMMARY OF THE INVENTION

It is a primary objective of the present invention to overcome the shortcomings of the prior art by providing a four-directional tip deflection device for endoscope that uses a bendable elastic element and a bendable four-directionally flexible body to provide a simple-structured four-directional tip deflection device for endoscope.

Another objective of the present invention is to provide a four-directional tip deflection device for endoscope that uses a flexible elastic element and a flexible four-directionally flexible body to prevent the bending angle of the elastic element and four-directionally flexible body from being interfered by the structure.

To achieve the foregoing objectives, the present invention provides a four-directional tip deflection device for endoscope comprising a tip deflecting mechanism, an elastic element, a support portion and at least one four-directionally flexible body. The tip deflecting mechanism includes an endoscope lens, first and second connecting portions facing with one another, and third and fourth connecting portions facing with one another. The elastic element is sheathed to the tip deflecting mechanism, and the elastic element includes a first accommodating space. The support portion is sheathed to the elastic element, and the support portion includes a second accommodating space. The four-directionally flexible body is disposed in the first accommodating space and among the first, second, third and fourth connecting portions, and an end of the four-directionally flexible body is connected to the tip deflecting mechanism, and another end of the four-directionally flexible body is connected to the support portion.

The first, second, third and fourth wires are connected to the first, second, third and fourth connecting portions respectively and threaded from the first accommodating space through the second accommodating space. If one of the first wire, second wire, third wire and fourth wire is pulled, the four-directionally flexible body will be bent into an angle in a corresponding first wire direction, second wire direction, third wire direction and fourth wire direction.

In summation of the description above, the four-directional tip deflection device for endoscope of the present invention has the following advantages:

(1) The four-directional tip deflection device for endoscope comes with simple structured elastic element and four-directionally flexible body to provide a low-priced four-directional tip deflection device for endoscope.

(2) The four-directional tip deflection device for endoscope comes with an elastic element capable of resuming the bent elastic element into its original position.

(3) The four-directional tip deflection device for endoscope uses flexible elastic element and four-directionally flexible body to provide a smaller radius of curvature for bending of the elastic element and the four-directionally flexible body.

(4) The four-directional tip deflection device for endoscope can control the bending direction and position of the lens more precisely.

### BRIEF DESCRIPTION OF THE DRAWINGS

FIG. 1 is a schematic view of a conventional four-directional tip deflection device for endoscope;

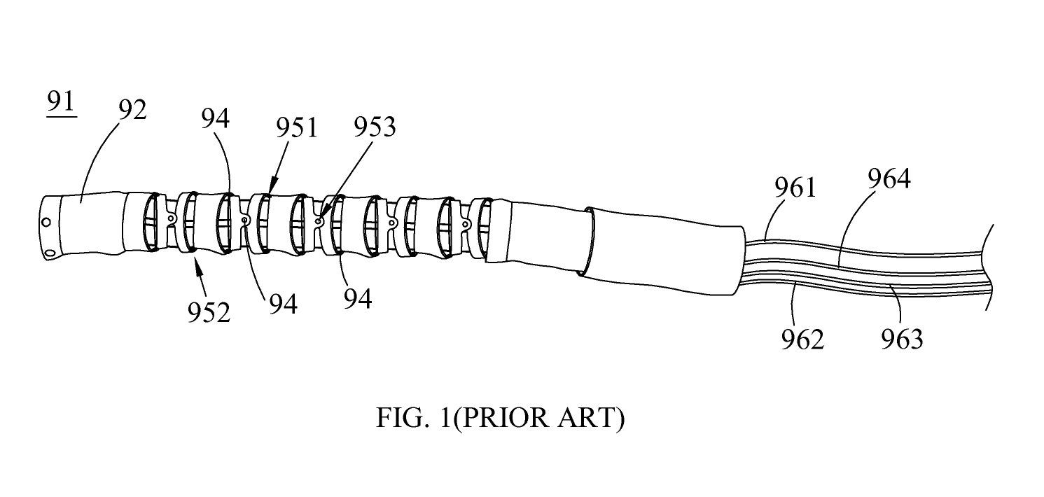

FIG. 2 is a schematic view of a four-directional tip deflection device for endoscope in accordance with a first embodiment of the present invention;

FIG. 3 is a side view of a four-directional tip deflection device for endoscope in accordance with a first embodiment of the present invention;

FIG. 4 is a rear view of a four-directional tip deflection device for endoscope in accordance with a first embodiment of the present invention;

FIG. 5 is a schematic view of a four-directional tip deflection device for endoscope in accordance with a second embodiment of the present invention;

FIG. 6 is a schematic view of a first movement of a four-directional tip deflection device for endoscope in accordance with a first embodiment of the present invention;

FIG. 7 is a schematic view of a second movement of a four-directional tip deflection device for endoscope in accordance with a first embodiment of the present invention;

FIG. 8 is a schematic view of a third movement of a four-directional tip deflection device for endoscope in accordance with a first embodiment of the present invention; and

FIG. 9 is a schematic view of a fourth movement of a four-directional tip deflection device for endoscope in accordance with a first embodiment of the present invention.

### DETAILED DESCRIPTION OF THE PREFERRED EMBODIMENTS

To make it easier for understanding the objective of the invention, its structure, innovative features, and performance, we use the embodiments together with the attached drawings for the detailed description of the invention. It is noteworthy to point out that same numerals are used for same respective elements in the illustration of the following embodiments.

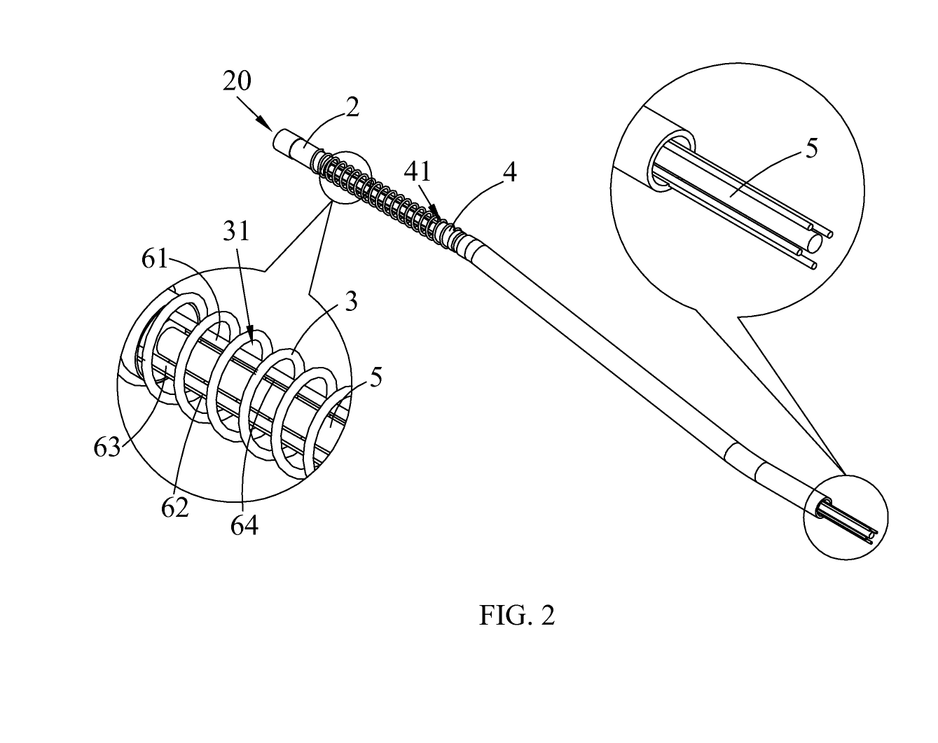

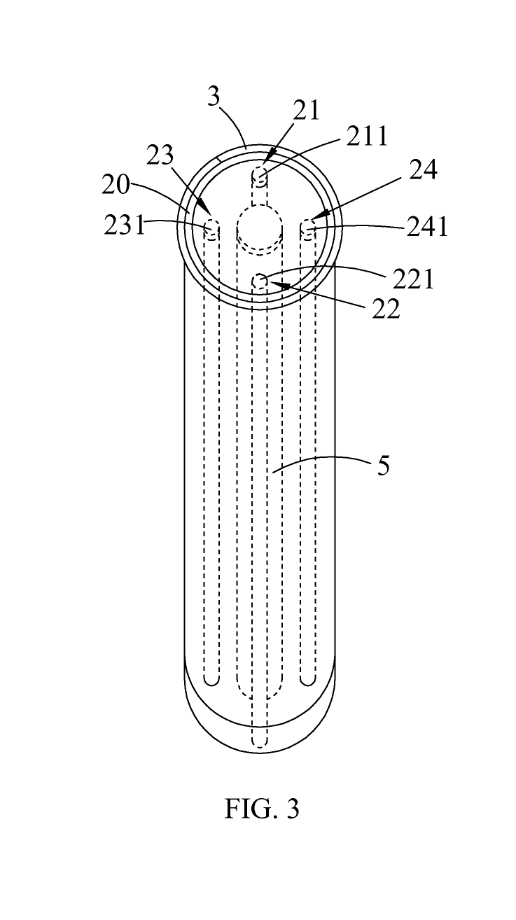

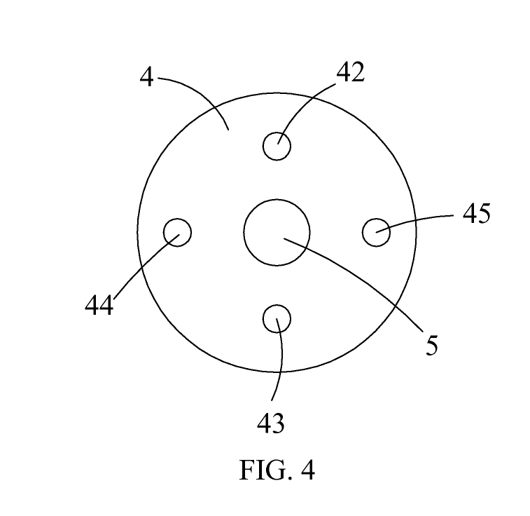

With reference to FIGS. 2 to 4 for a schematic view, a side view and a rear view of a four-directional tip deflection device for endoscope in accordance with a first embodiment of the present invention respectively, the four-directional tip deflection device for endoscope 1 comprises a tip deflecting mechanism 2, an elastic element 3, a support portion 4, at least one four-directionally flexible body 5, and a first wire 61, a second wire 62, a third wire 63 and a fourth wire 64. The tip deflecting mechanism 2 includes an endoscope lens 20, a first connecting portion 21 and a second connecting portion 22 facing with one another, and a third connecting portion 23 and a fourth connecting portion 24 facing with one another, wherein the first connecting portion 21 can be a first connecting hole 211, and the second connecting portion 22 can be a second connecting hole 221, and the third connecting portion 23 can be a third connecting hole 231, and the fourth connecting portion 24 can be a fourth connecting hole 241, or the four connecting portions can be connected to each other by a soldering method (not shown in the figure) according to the actual requirements to provide a way of latching the first wire 61, second wire 62, third wire 63 and fourth wire 64 into the first connecting hole 211, second connecting hole 221, third connecting hole 231 and fourth connecting hole 241 respectively.

The elastic element 3 is a spring sheathed onto the tip deflecting mechanism 2, and the elastic element 3 includes a first accommodating space 31 therein. The support portion 4 is sheathed to the elastic element 3, and the support portion 4 includes a second accommodating space 41 disposed therein, a first penetrating hole 42 and a second penetrating hole 43 facing with one another, and a third penetrating hole 44 and a fourth penetrating hole 45 facing with one another, wherein the first penetrating hole 42 is disposed on a same side of the first connecting portion 21, and the second penetrating hole 43 is disposed on a same side of the second connecting portion 22, and the third penetrating hole 44 is disposed on a same side of the third connecting portion 23, and the fourth penetrating hole 45 is disposed on a same side of the fourth connecting portion 24. The tip deflecting mechanism 2 and the support portion 4 are metal rings.

At least one four-directionally flexible body 5 is made of a four-directionally flexible metal material or plastic material as needed, wherein the four-directionally flexible body 5 is in a four-directionally flexible rectangular shape, irregular shape or circular cylindrical shape. In this embodiment, the four-directionally flexible body 5 is in the four-directionally flexible circular cylindrical shape. The four-directionally flexible body 5 is disposed in the first accommodating space 31 and among the first connecting portion 21, second connecting portion 22, third connecting portion 23 and fourth connecting portion 24. The four-directionally flexible body 5 can be installed on one side, at the center or on both sides in the first accommodating space 31, and an end of the four-directionally flexible body 5 is fixed to the tip deflecting mechanism 2, and another end of the four-directionally flexible body 5 is fixed to the support portion 4 to prevent the four-directionally flexible body 5 from rotating with respect to itself as being an axis.

The first wire 61, second wire 62, third wire 63 and fourth wire 64 are either metal wires or plastic wires connected to the first connecting portion 21, second connecting portion 22, third connecting portion 23 and fourth connecting portion 24 respectively, and threaded from the first accommodating space 31 through the second accommodating space 41, wherein the first wire 61 and the second wire 62 are threaded through the first penetrating hole 42 and the second penetrating hole 43 respectively, and the third wire 63 and the fourth wire 64 are threaded through the third penetrating hole 44 and the fourth penetrating hole 45 respectively to control a bending direction of the four-directionally flexible body 5.

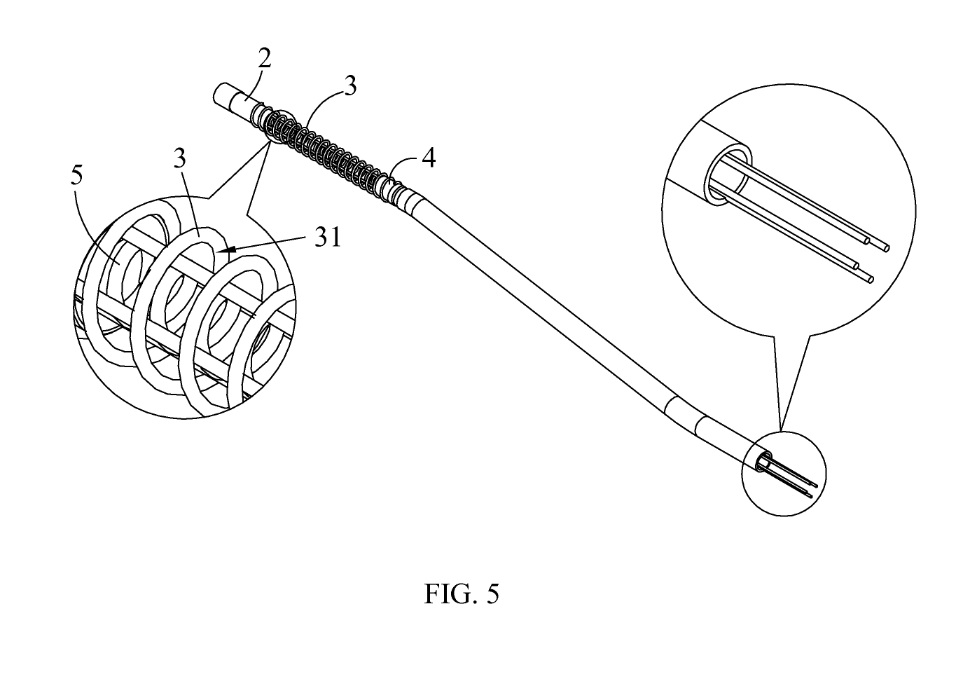

With reference to FIG. 5 for a schematic view of a four-directional tip deflection device for endoscope in accordance with a second embodiment of the present invention, the structure of the second embodiment is substantially the same as the first embodiment, and the difference resides on that the four-directionally flexible body 5 of the second embodiment is a spring instead of a metal pillar or plastic pillar as used in the four-directionally flexible body of the first embodiment. In the second embodiment, an end of the four-directionally flexible body 5 is connected to the tip deflecting mechanism 2, and another end of the four-directionally flexible body 5 is connected to the support portion 4 for supporting and moving a tip deflecting mechanism at an end of the four-directionally flexible body 5.

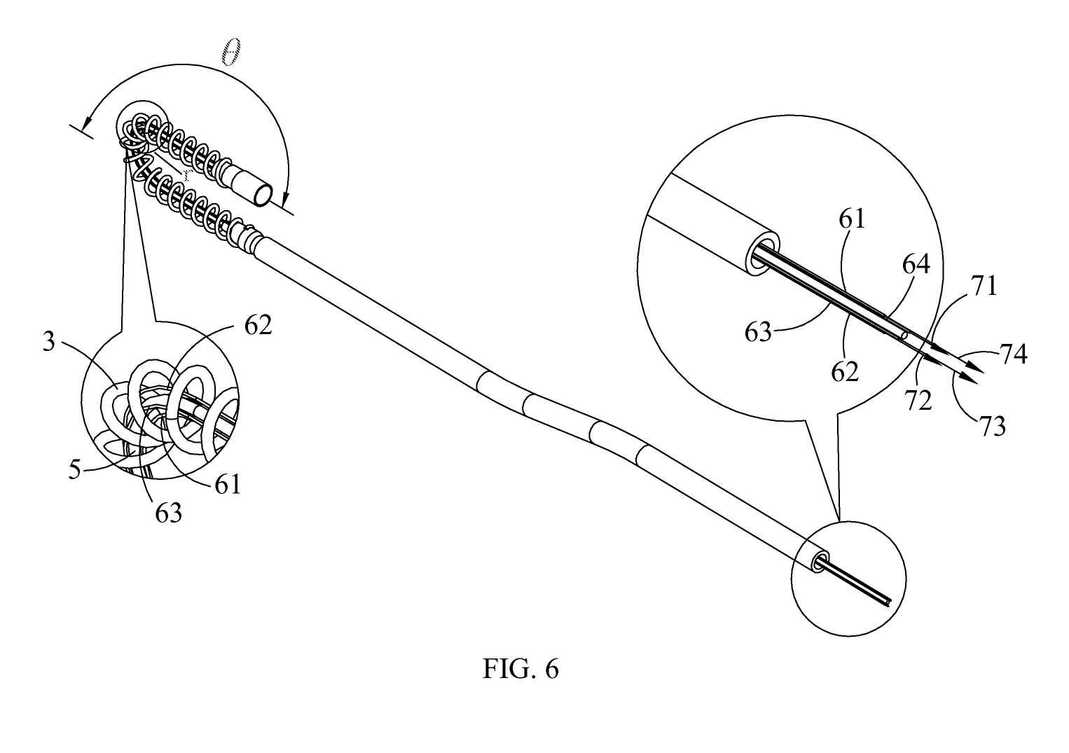

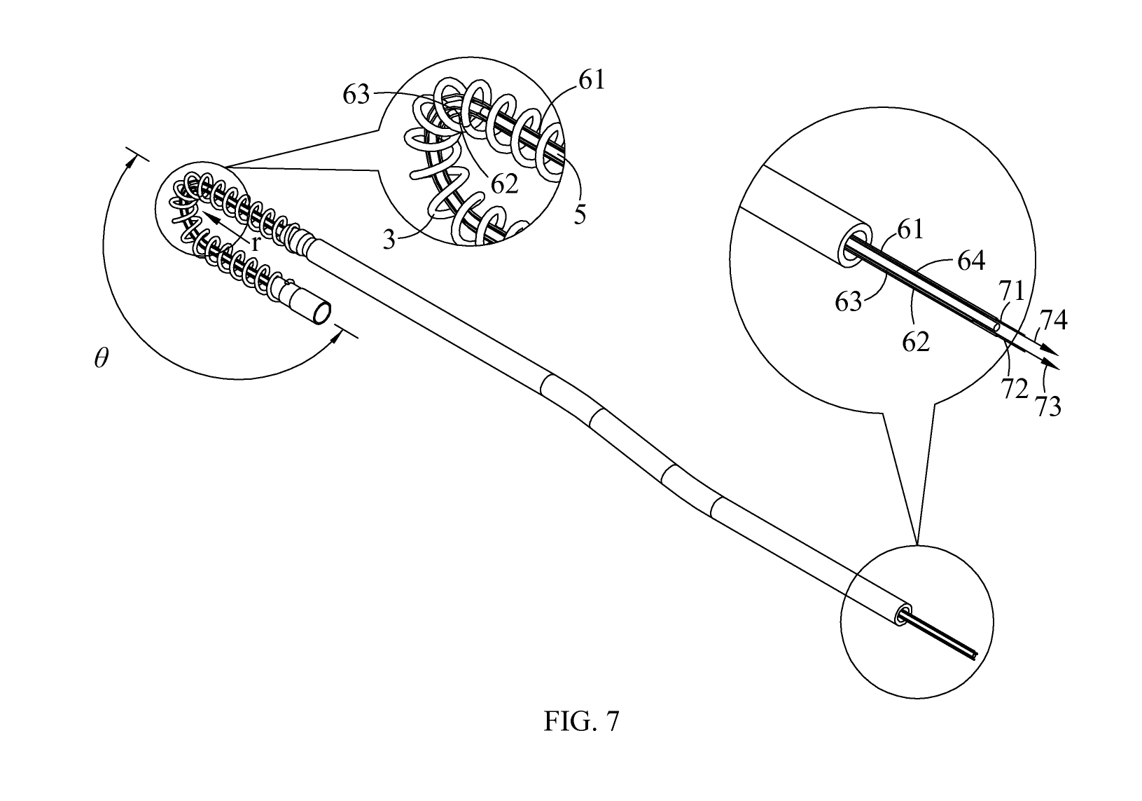

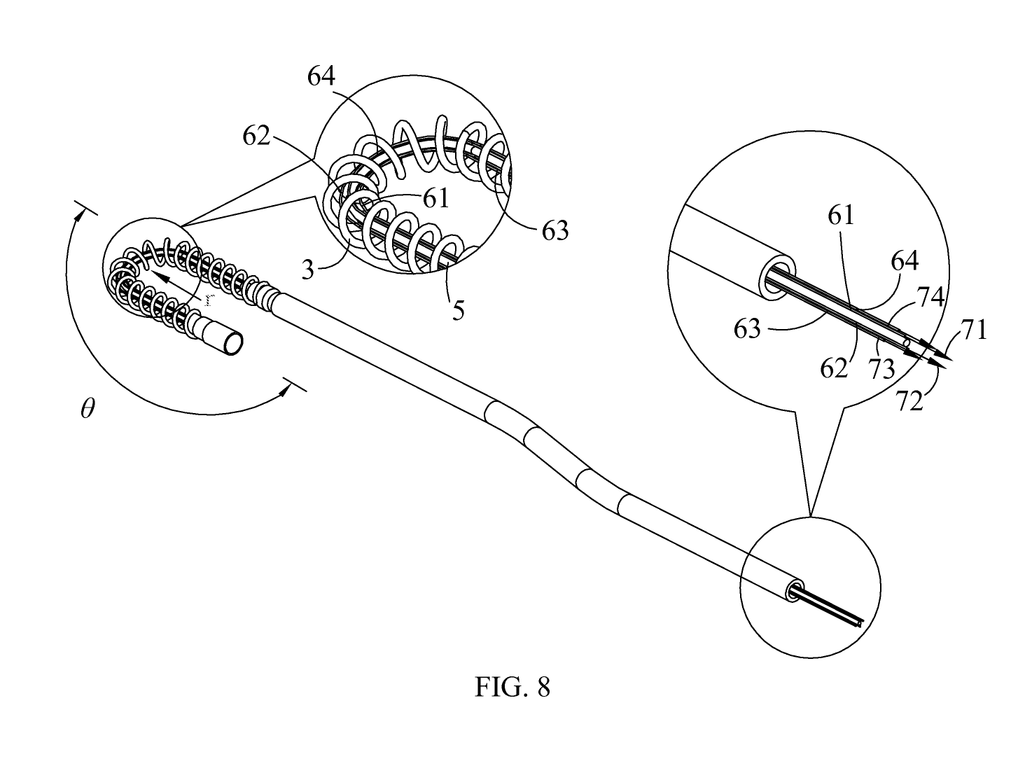

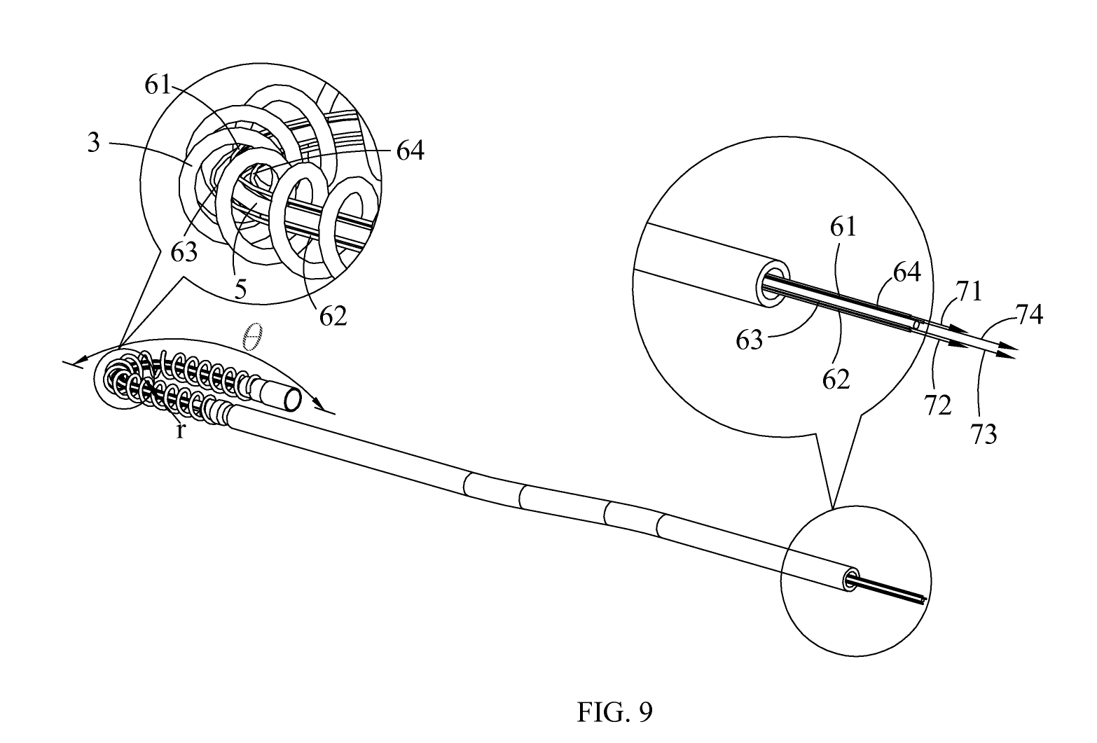

With reference to FIGS. 6 to 9 for schematic views of a first movement, a second movement, a third movement and a fourth movement of a four-directional tip deflection device for endoscope in accordance with a first embodiment of the present invention respectively, if one of the first wire 61, second wire 62, third wire 63 and fourth wire 64 connected to the tip deflecting mechanism 2 is pulled, the elastic element 3 and the four-directionally flexible body 5 will be bent into an angle θ from 0 degree to 180 degrees corresponding with a first wire direction 71, a second wire direction 72, a third wire direction 73 or a fourth wire direction 74 respectively. By bending the four-directionally flexible body 5 and the elastic element 3 with a smaller radius of curvature r towards the first wire direction 71, second wire direction 72, third wire direction 73 or fourth wire direction 74, the invention can achieve the effects of observing a complete tissue image inside the cavity, preventing any missing tissue image in the cavity, and avoiding limitations on the application of the four-directional tip deflection device for endoscope 1. If the pulled first wire 61, second wire 62, third wire 63 or fourth wire 64 is released and loosened, the tip deflecting mechanism 2 together with the bent elastic element 3 will return to their original positions without remaining at the pulled angle or position, so as to overcome the inconvenient use of the conventional four-directional tip deflection device for endoscope.

While the invention has been described by means of specific embodiments, numerous modifications and variations could be made thereto by those skilled in the art without departing from the scope and spirit of the invention set forth in the claims.

What is claimed is:

1. A four-directional tip deflection device for endoscope, comprising:
a tip deflecting mechanism, including an endoscope lens, a first connecting portion and a second connecting portion facing with one another, and a third connecting portion and a fourth connecting portion facing with one another;
an elastic element, coupled to the tip deflecting mechanism, and including a first accommodating space;
a support portion, coupled to the elastic element, and including a second accommodating space;
at least one four-directionally flexible body, disposed in the first accommodating space, and an end of the four-directionally flexible body being fixed to the tip deflecting mechanism, and another end of the four-directionally flexible body being fixed to the support portion; and
a first wire, a second wire, a third wire and a fourth wire, coupled to the first connecting portion, the second connecting portion, the third connecting portion and the fourth connecting portion respectively, and threaded from the first accommodating space through the second accommodating space;
wherein, if one of the first wire, the second wire, the third wire and the fourth wire is pulled, the four-directionally flexible body will be bent into an angle corresponding with a first wire direction, a second wire direction, a third wire direction and a fourth wire direction respectively.

2. The four-directional tip deflection device for endoscope of claim 1, wherein the first, second, third and fourth connecting portions are fixed to an end of the first, second, third and fourth wires respectively.

3. The four-directional tip deflection device for endoscope of claim 1, wherein the support portion includes a first penetrating hole and a second penetrating hole facing with one another, and a third penetrating hole and a fourth penetrating hole facing with one another, and the first and second penetrating holes are disposed on a same side of the first and second connecting portions respectively, and the third and fourth penetrating holes are disposed on a same side of the third and fourth connecting portions respectively.

4. The four-directional tip deflection device for endoscope of claim 3, wherein the first and second wires are threaded into the first and second penetrating holes respectively, and the third and fourth wires are threaded through the third and fourth penetrating holes respectively, for controlling a bending direction of the four-directionally flexible body.

5. The four-directional tip deflection device for endoscope of claim 1, wherein the four-directionally flexible body is made of a four-directionally flexible metal material or plastic material.

6. The four-directional tip deflection device for endoscope of claim 1, wherein the elastic element is a spring, a soft tube or a braided tube made of a flexible material for wrapping the four-directionally flexible body and maintaining a tubular structure of the four-directional flexible body.

7. The four-directional tip deflection device for endoscope of claim 1, wherein the tip deflecting mechanism and the support portions are metal rings.

8. The four-directional tip deflection device for endoscope of claim 1, wherein the first, second, third and fourth wires are metal wires or plastic wires.

9. The four-directional tip deflection device for endoscope of claim 1, wherein the four-directionally flexible body is flexible in four directions, and in a rectangular shape, an irregular shape or a circular cylindrical shape.
